(19) United States
(12) Patent Application Publication
(10) Pub. No.: US 2006/0242772 A1
(43) Pub. Date: Nov. 2, 2006

(54) Reductive colouring system for keratin fibres

(76) Inventors: Markus Speckbacher, Aschaffenburg (DE); Jessica Chassot, Chavannes-sous-Orsonnens (CH); Hans-Jurgen Braun, Ueberstorf (CH)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/395,791

(22) Filed: Mar. 31, 2006

(30) Foreign Application Priority Data
Apr. 2, 2005 (EP) 05007230.5

(51) Int. Cl. A61K 8/00

(52) U.S. Cl. 8/405

(57) ABSTRACT

Agent for colouring keratin-containing fibres, in particular human hair, comprising: a) ascorbic acid; b) at least one cationic oxime-ester; and c) at least one reactive carbonyl compound, as well as method of colouring hair using this agent.

The present invention relates to a reductive colouring system consisting of oxime compounds and carbonyl compounds, and ascorbic acid as reducing agent, and to colorants comprising these compounds for keratin fibres, such as, for example, human hair, wool or furs.

In general, two processes are used for colouring keratin-containing fibres. One possibility consists in the use of direct dyes. These are incorporated into suitable carrier masses in order then to be applied to the fibres. This method, generally known as tinting, is easy to use, exceptionally mild and is characterized by low damage to the keratin fibres since no ammonia or peroxide is added. However, the durability and wash resistance of this colouring method is generally unsatisfactory, for which reason a direct method is also referred to as semipermanent hair colour.

A long-lasting coloration, also called permanent hair colour, can be produced with oxidation dyes which are formed by oxidative coupling of one or more developer components with one or more coupler components. If required, oxidation-stable direct dyes can also be added to the oxidative system in order to achieve particular colour effects. When using oxidizing agents, however, damage to the hair structure is observed. Furthermore, some of the oxidation hair colour precursors used (in particular aromatic amines) have a considerable sensitization potential, as a result of which skin irritations may arise in people with a corresponding disposition.

An object of the present invention is therefore to provide a permanent hair colouring system consisting of oxime compounds and carbonyl compounds with which the colorations achieved can on the one hand be applied gently to the fibres, and which is acceptable from a toxicological and dermatological point of view, and on the other hand also permits an intense and brilliant colour result. Furthermore, the resulting hair coloration must have good light fastness and resistance to shampooing (durability of the coloration) and a good rubbing fastness.

Surprisingly, it has now been found that fashionable and luminous colour shades can be produced on keratin-containing fibres if a reductive colorant is applied to the fibres which comprises certain cationic oxime-ester compounds, reactive carbonyl compounds and ascorbic acid (vitamin C). The water soluble cationic oxime-ester compounds are reduced to the corresponding amines (the cationic part is removed during the reduction process), which in turn react straightaway with the reactive carbonyl compounds in the hair to give coloured substances. The resulting colorations are extremely resistant to washing and durable. Although of course no hydrogen peroxide (oxidizing agent!) can be used during the reduction process, lightening of the natural colour shade, if this is desired, is possible but not necessarily required in the course of a pretreatment.

The cationic oxime-ester compounds according to the invention can be produced by nitrosation of CH-acidic compounds, followed by esterification of the oxime group and quaternization of the ester group with N-nucleophiles, affording the desired cationic material. CH-acidic compounds are generally regarded as being those compounds which carry a hydrogen atom bonded to an aliphatic carbon atom where, on account of the electron-withdrawing substituents, activation of the corresponding carbon-hydrogen bond is effected.

Reactive carbonyl compounds according to the invention have at least one carbonyl group as reactive group.

The present invention therefore provides an agent for colouring keratin-containing fibres, in particular human hair, which is characterized in that it comprises a) ascorbic acid (vitamin C) as reducing agent, b) at least one cationic oxime-ester compound of the formula A1-A10 as “developer substance” and c) at least one reactive carbonyl compound of the formula B1-B12 as “coupler substance”.

The oxime compounds A1-A10 according to the invention are defined as follows:

Compounds with the formula A1: 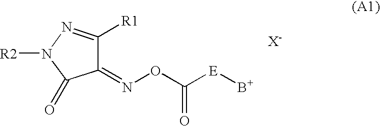
in which R1 and R2 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group, a substituted or unsubstituted aminoaryl group, where R1 can likewise be a C₁-C₆-alkoxy group. E may be a C₁-C₄-alkyl bridge, a C₃-C₆-cycloalkyl bridge or an aryl bridge. B⁺ may be
a) an aromatic heterocyclic ammonium compound, preferably a cationic derivative of N-methyl-imidazole, N-allyl-imidazole, 2-ethyl-imidazole or 1,2-dimethyl-imidazole or a cationic derivative of pyridine, 4-dimethylamino-pyridine, pyrimidine, pyrazole, N-methyl-pyrazole or chinoline; or
b) a non-aromatic heterocyclic ammonium compound, in particular a cationic derivative of N-methyl-morpholine, N-ethyl-morpholine or 1-methyl-piperidine; or
c) an alkylammonium compound or an arylammonium compound according to the formula NRₐR_{b}R_{c}, in which Rₐ, R_{b} and R_{c}, independently of one other, are a benzyl rest, a phenyl rest or a C₁-C₆-alkyl rest, preferably a methyl group, an ethyl group, a propyl group, an isopropyl group or a butyl group, whereas the prementioned alkyl groups may be unsubstituted or substituted with one or more hydroxy groups or amino groups; or
d) a cationic phosphonium group, e.g., a tributyl phosphonium group, preferably a trimethyl phosphonium group or a triethyl phosphonium group.

Compounds with the formula A2: 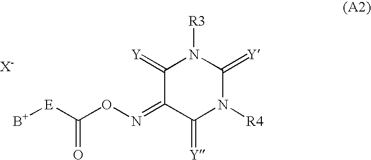
in which R3 and R4 may be identical or different and, independently of one another, are a hydrogen atom or a substituted or unsubstituted C₁-C₆-alkyl group;
Y, Y′ and Y″, independently of one another, are an oxygen atom, a sulphur atom or an NH group. E and B⁺ have the abovementioned meaning.

Compounds with the formula A3: 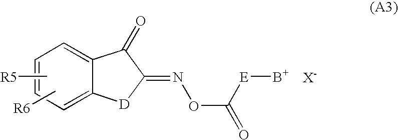
in which D is an oxygen atom, a sulphur atom, a sulphoxyl group, a sulphonyl group or a group N—R₅ₐ, where R₅ₐ is a hydrogen atom or a substituted or unsubstituted C₁-C₆-alkyl group;
and R5 and R6, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR7R8, where R7 and R8, independently of one another, are a hydrogen atom or a C₁-C₆-alkyl group.
E and B⁺ have the abovementioned meaning.

Compounds with the formula A4 or A5: 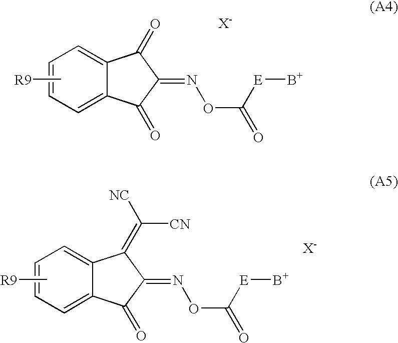
in which R9 is a hydrogen atom, a halogen atom, a hydroxyl group, a nitro group, a C₁-C₆-alkyl group, a C₁-C₆-alkoxy group, a carboxyl group, a carboxamide group, a cyano group or an amino group —NR10R11, where R10 and R11, independently of one another, are a hydrogen atom or a C₁-C₆-alkyl group.
E and B⁺ have the abovementioned meaning.

Compounds with the formula A6: 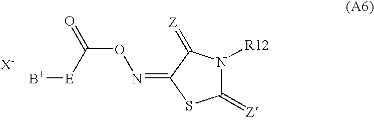
in which Z is an oxygen atom or a group —NR13, where R13 is a hydrogen atom or a C₁-C₆-alkyl group;
Z′ is a sulphur atom or a group —NR14, where R14 is a hydrogen atom or a C₁-C₆-alkyl group; and
R12 is a hydrogen atom, a C₁-C₆-alkyl group or a C₁-C₄-carboxyalkyl group.
E and B⁺ have the abovementioned meaning.

Compounds with the formula A7: 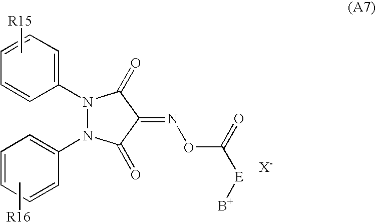
in which R15 and R16 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR17R18, where R17 and R18, independently of one another, are a hydrogen atom or a C₁-C₆-alkyl group.
E and B⁺ have the abovementioned meaning.

Compounds with the formula A8 or A9: 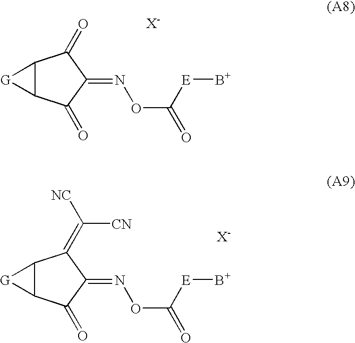
in which G is a fused, substituted or unsubstituted aromatic or heteroaromatic ring to which a further aromatic or heteroaromatic ring may additionally be fused.
E and B⁺ have the abovementioned meaning.

Compounds of the formula A10: 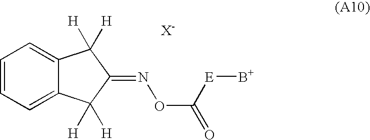
in which E and B+have the abovementioned meaning.

The counterions X⁻ used are preferably sulphate anions, methylsulphate anions, phosphate anions, hydrogenphosphate anions, oxalate anions, formate anions, acetate anions, citrate anions, tartrate anions, malonate anions, pyruvate anions or halogen anions, particular preference being given to the chloride anion, bromide anion and methylsulphate anion.

The cationic oxime-ester compounds (developer substances) of the general formula A1-A10 are preferably chosen from

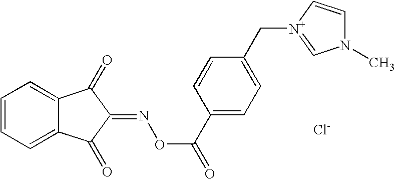

### 3-[4-({[(1,3-dioxo-1,3-dihydro-2H-inden-2-ylidene)amino]oxy}carbonyl)-benzyl]-1-methyl-1H-imidazol-3-ium chloride

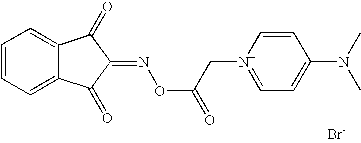

### 4-(dimethylamino)-1-(2-{[(1,3-dioxo-1,3-dihydro-2H-inden-2-ylidene)-amino]oxy}-2-oxoethyl)pyridinium bromide

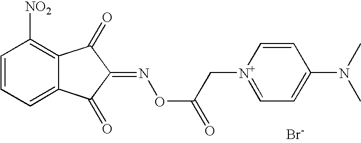

### 4-(dimethylamino)-1-[2-({[(2E)-4-nitro-1,3-dioxo-1,3-dihydro-2H-inden-2-ylidene]amino}oxy)-2-oxoethyl]pyridinium bromide

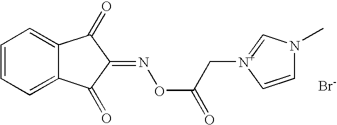

### 3-(2-{[(1,3-dioxo-1,3-dihydro-2H-inden-2-ylidene)amino]oxy}-2-oxoethyl)-1-methyl-1H-imidazol-3-ium bromide

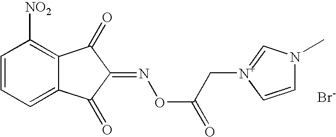

### 1-methyl-3-[2-({[(2E)-4-nitro-1,3-dioxo-1,3-dihydro-2H-inden-2-ylidene]-amino}oxy)-2-oxoethyl]-1H-imidazol-3-ium bromide

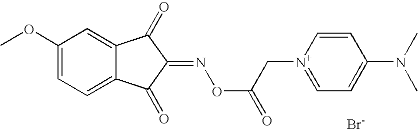

### 4-(dimethylamino)-1-[2-({[(2E)-5-methoxy-1,3-dioxo-1,3-dihydro-2H-inden-2-ylidene]amino}oxy)-2-oxoethyl]pyridinium bromide

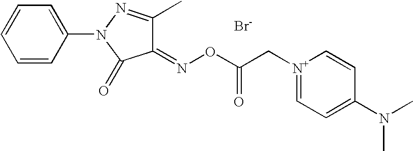

### 4-(dimethylamino)-1-[2-({[(4E)-3-methyl-5-oxo-1-phenyl-1,5-dihydro-4H-pyrazol-4-ylidene]amino}oxy)-2-oxoethyl]pyridinium bromide

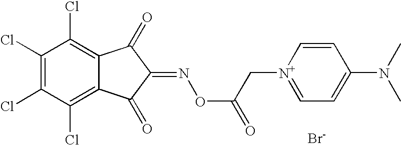

### 4-(dimethylamino)-1-(2-oxo-2-{[(4,5,6,7-tetrachloro-1,3-dioxo-1,3-dihydro-2H-inden-2-ylidene)amino]oxy}ethyl)pyridinium bromide

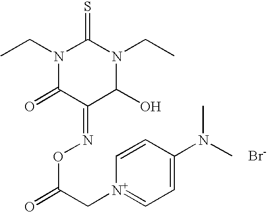

### 1-(2-{[((5Z)-1,3-diethyl-4-hydroxy-6-oxo-2-thioxotetrahydro-5 (2H)-pyrimidinylidene)amino]oxy}-2-oxoethyl)-4-(dimethylamino)pyridinium bromide

The carbonyl compounds B1 to B12 according to the invention are defined as follows:

Compounds with the formula B1: 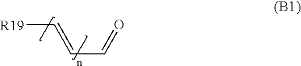
in which R19 is a substituted or unsubstituted aryl group to which a further aromatic ring may optionally also be fused, or a substituted or unsubstituted, saturated or unsaturated heterocycle; and n is 0, 1, 2 or 3.

Compounds with the formula B2: 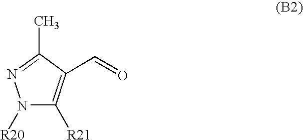
in which R20 is a C₁-C₆-alkyl group, a substituted or unsubstituted aryl radical or a substituted or unsubstituted, saturated or unsaturated heterocycle; and
R21 is a pyrrole radical, an imidazole radical, a pyrazole radical, an indole radical, a pyrrolidine radical, a morpholine radical, a dimethylamine radical, a phenol radical or a thiophenol radical, where these radicals are in each case bonded to the general formula B2 via the heteroatom.

Pyridoxal (vitamin B₆) with the formula B3:

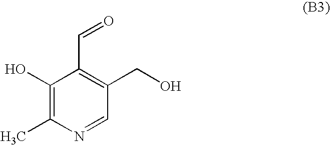

All-trans-retinal (vitamin A) with the formula B4:

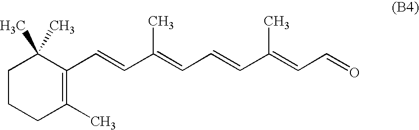

Compounds with the formula B5: 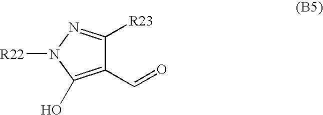
in which R22 and R23 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group or a substituted or unsubstituted aminoaryl group, and R22 can likewise be a C₁-C₆-alkoxy group.

Compound with the formula B6 or B7: 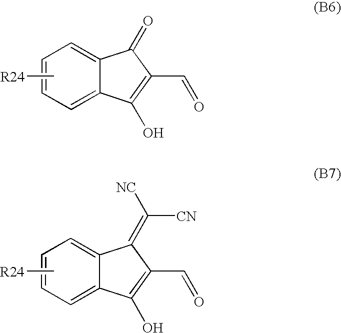
in which R24 is a hydrogen atom, a halogen atom, a hydroxyl group, a nitro group, a C₁-C₆-alkyl group, a C₁-C₆-alkoxy group, a carboxyl group, a carboxamide group, a cyano group or an amino group —NR25R26, where R25 and R26, independently of one another, are a hydrogen atom or a C₁-C₆-alkyl group.

Compounds with the formula B8 or B9: 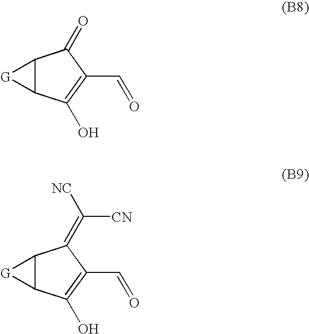
in which G has the abovementioned meaning.

Compounds with the formula B10 or B11: 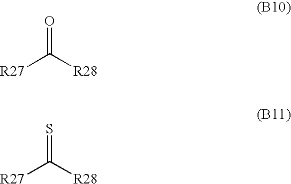
in which R27 and R28 may be identical or different and, independently of one another, are a C₁-C₆-alkyl group or a substituted or unsubstituted aryl group.

Compounds with the formula B12: 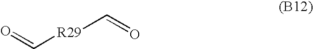
in which R29 is a substituted or unsubstituted, saturated or unsaturated C₁-C₆-alkyl bridge or a substituted or unsubstituted aryl bridge.

The reactive carbonyl compounds (coupler substances) of the general formulae B1 to B12 are preferably chosen from

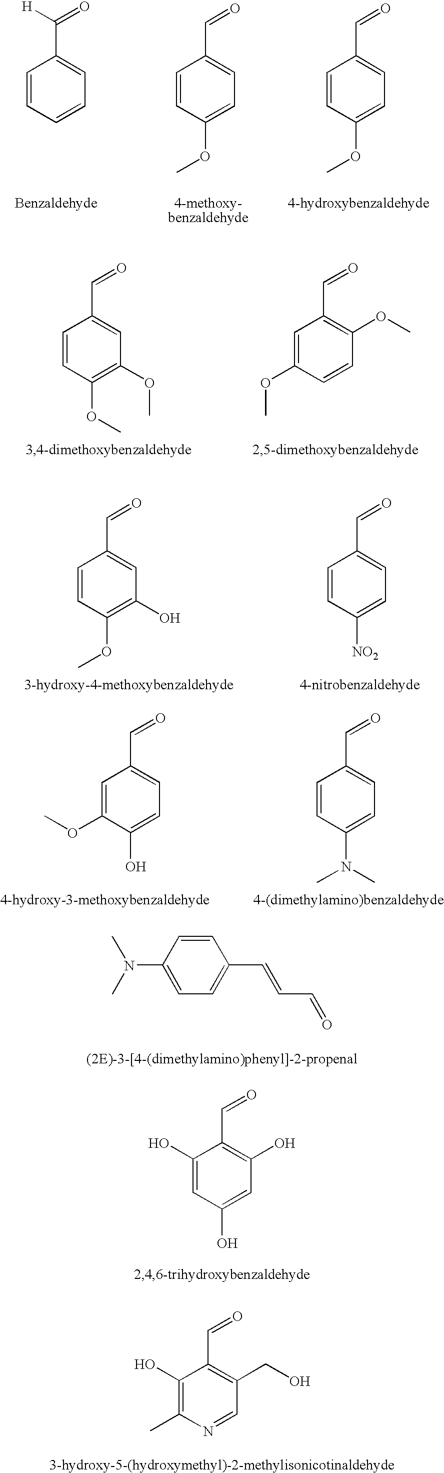

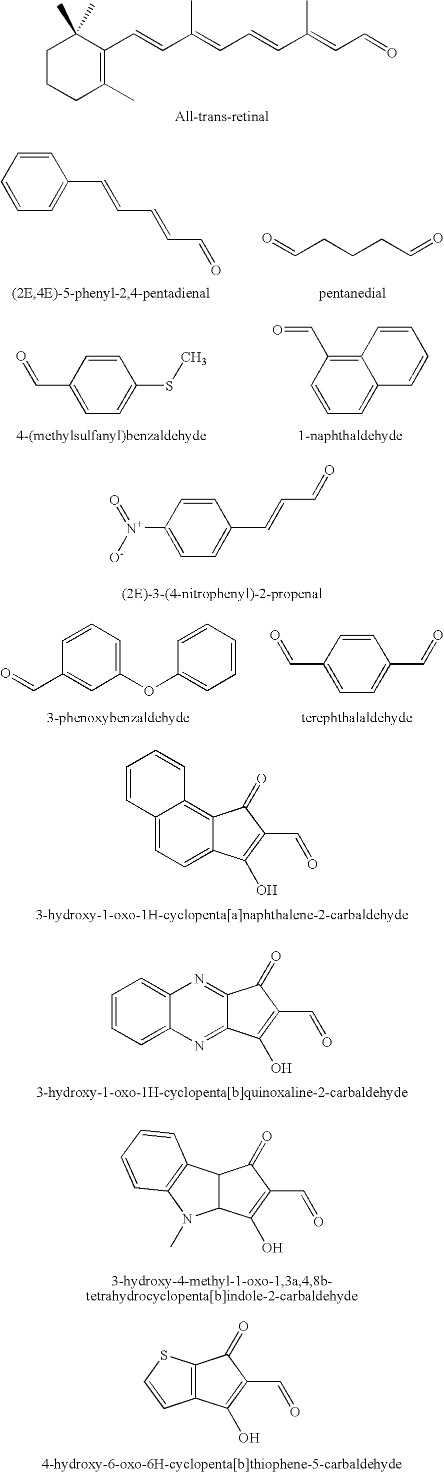

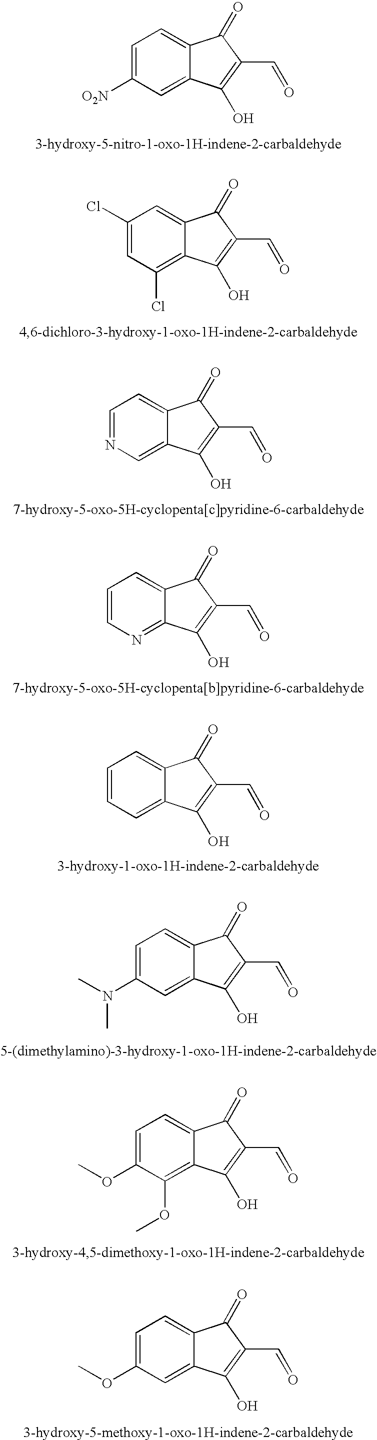

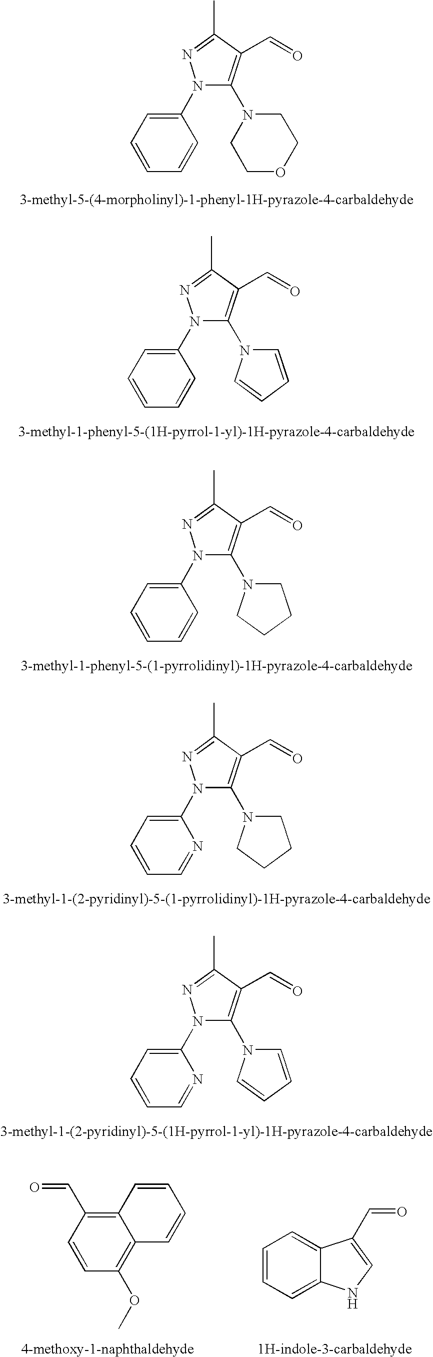

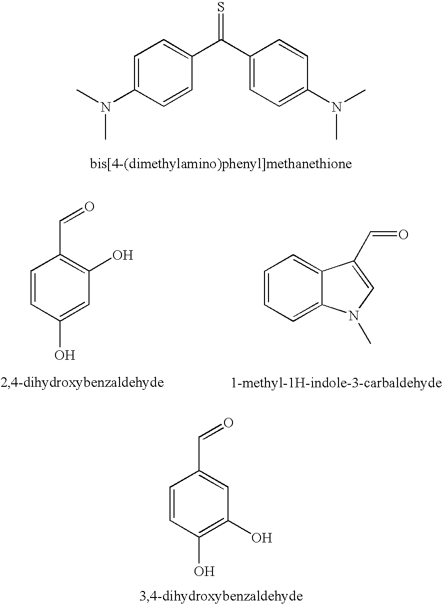

The cationic oxime-ester compounds according to the invention of the general formula A1 to A10 can be prepared via a three step synthesis starting from CH acidic compounds such as substituted or unsubstituted 1,3-indanediones. These are either available commercially or are accessible through standard operations from components which are commercially available or can be prepared easily in accordance with literature procedures (e.g., G. Sartori et al., J. Chem. Soc. Perkin. Trans. 1, 1992, 2985-2988 and G. Vanag et. al. J. of General Chemistry of the U.S.S.R. 28(6), 1958, 1570-1572). The first step involves a nitrosation reaction of the CH-acidic starting compound yielding the oxime derivate. Subsequent condensation with aliphatic or aromatic carbonacid chlorides/bromides or halogeno-carbonacid anhydrides converts the oxime to the corresponding ester derivative (scheme 1).

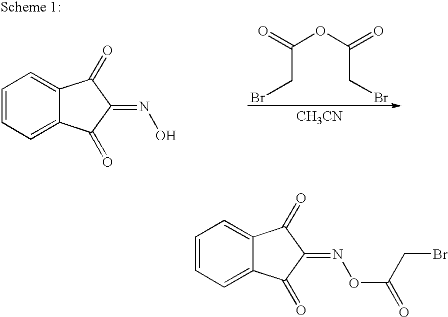

This ester is not isolated and reacts in turn straightaway with aliphatic or aromatic N-nucleophiles (e.g., 4-dimethylamino-pyridine) to afford the desired water soluble cationic oxime-ester (scheme 2).

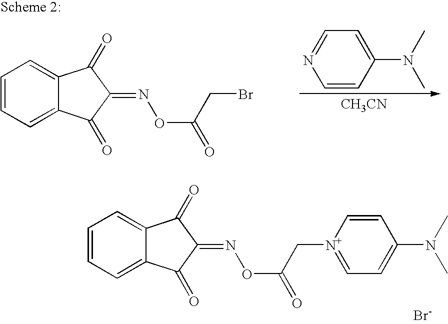

During the dyeing procedure, the cationic oxime-ester compounds according to the invention of the general formula A1-A10 are reduced to the corresponding amines while the cationic part is cleaved by hydrolysis effects (scheme 3).

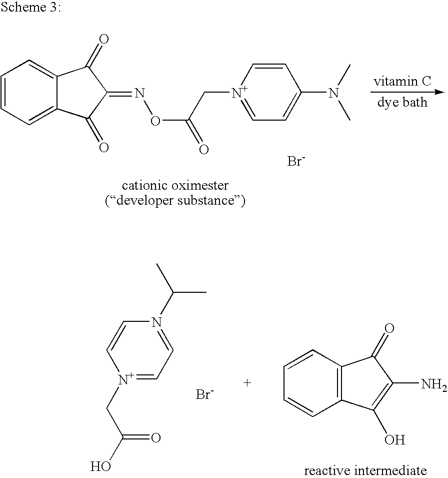

The obtained amines are very reactive and react very rapidly with the carbonyl compounds according to the invention of the general formula B1-B12 to give the corresponding colored compounds (azomethines or Schiff's bases) (scheme 4).

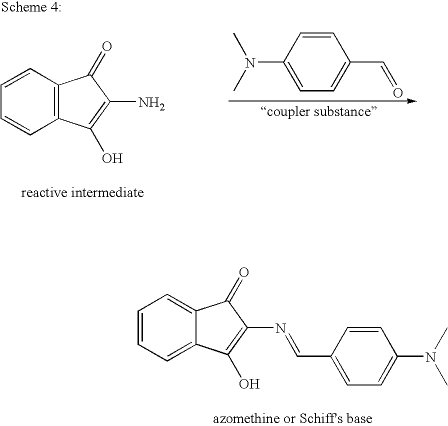

The oxime compounds according to the invention of the general formula A1-A10 and the carbonyl compounds according to the invention of the general formula B1-B12 are present in the colorants according to the invention preferably in a total amount of from 0.1 to 10 percent by weight, in particular 0.5 to 8 percent by weight.

To produce special colour nuances, besides the components used according to the invention, it is possible to additionally also add to the agents according to the invention one or more customary direct dyes from the group of acidic and basic dyes, nitro dyes, azo dyes, anthraquinone dyes and triphenylmethane dyes, such as for example, 1,4-bis[(2-hydroxyethyl)amino]-2-nitrobenzene, 1-(2-hydroxyethyl)amino-2-nitro-4-[di-(2-hydroxyethyl)amino]benzene, (HC Blue No. 2), 1-amino-3-methyl-4-[(2-hydroxyethyl)amino]-6-nitrobenzene, (HC Violet No. 1), 4-[ethyl-(2-hydroxyethyl)amino]-1-[(2-hydroxyethyl)amino]-2-nitrobenzene hydrochloride (HC Blue No. 12), 1-[(2,3-dihydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitrobenzene hydrochloride (HC Blue No. 9), 1-(3-hydroxypropylamino)-4-[di(2-hydroxyethyl)amino]-2-nitrobenzene, (HC Violet No. 2); 1-amino-4-[(2-hydroxyethyl)amino]-2-nitrobenzene (HC Red No. 7), 2-amino-4,6-dinitrophenol, 1,4-diamino-2-nitrobenzene (CI76070), 4-amino-2-nitrodiphenylamine (HC Red No. 1), 1-amino-4-[di(2-hydroxyethyl)amino]-2-nitrobenzene hydrochloride (HC Red No. 13), 1-amino-5-chloro-4-[(2-hydroxyethyl)-amino]-2-nitrobenzene, 4-amino-1-[(2-hydroxyethyl)amino]-2-nitrobenzene (HC Red No. 3), 4-amino-2-nitro-1-((prop-2-en-1-yl)amino)-benzene, 4-amino-3-nitrophenol, 4[(2-hydroxyethyl)amino]-3-nitrophenol, 4-[(2-nitrophenyl)amino]phenol (HC Orange No. 1), 1-[(2-aminoethyl)-amino]-4-(2-hydroxyethoxy)-2-nitrobenzene (HC Orange No. 2), 4-(2,3-dihydroxypropoxy)-1-[(2-hydroxyethyl)amino]-2-nitrobenzene, (HC Orange No. 3), 1-amino-5-chloro-4-[(2,3-dihydroxypropyl)amino]-2-nitrobenzene (HC Red No. 10), 5-chloro-1,4-[di(2,3-dihydroxypropyl)-amino]-2-nitrobenzene (HC Red No. 11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 3-amino-6-(methylamino)-2-nitropyridine, 1,2,3,4-tetrahydro-6-nitroquinoxaline, 7-amino-3,4-dihydro-6-nitro-2H-1,4-benzoxazine (HC Red No. 14), 1,2-diamino-4-nitrobenzene (CI76020), 1-amino-2-[(2-hydroxyethyl)amino]-5-nitrobenzene (HC Yellow No. 5), 1-(2-hydroxyethoxy)-2-[(2-hydroxyethyl)amino]-5-nitrobenzene, (HC Yellow No. 4), 1-[(2-hydroxyethyl)amino]-2-nitrobenzene (HC Yellow No. 2), 2-[(2-hydroxyethyl)amino]-1-methoxy-5-nitrobenzene, 2-amino-3-nitrophenol, 1-amino-2-methyl-6-nitrobenzene, 1-(2-hydroxyethoxy)-3-methylamino-4-nitrobenzene, 2,3-(dihydroxypropoxy)-3-methylamino-4-nitrobenzene, 2-[(2-hydroxyethyl)amino]-5-nitrophenol (HC Yellow No. 11), 3-[(2-aminoethyl)amino]-1-methoxy-4-nitrobenzene hydrochloride, (HC Yellow No. 9), 1-[(2-ureidoethyl)amino]-4-nitrobenzene, 4-[(2,3-dihydroxypropyl)amino]-3-nitro-1-trifluoromethylbenzene, (HC Yellow No. 6), 1-chloro-2,4-bis[(2-hydroxyethyl)amino]-5-nitrobenzene (HC Yellow No. 10), 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hydroxyethyl)amino]-3-nitro-1-trifluoromethylbenzene, (HC Yellow No. 13), 4-[(2-hydroxyethyl)amino]-3-nitrobenzonitrile (HC Yellow No. 14), 4-[(2-hydroxyethyl)amino]-3-nitrobenzamide (HC Yellow No. 15), 2,4-dinitro-1-hydroxynaphthalene, 1,4-di [(2,3-dihydroxypropyl)amino]-9,10-anthraquinone, 1,4-di [(2-hydroxyethyl)amino]-9,10-anthraquinone (CI61545, Disperse Blue 23), 1-amino-4-hydroxy-9,10-anthraquinone (CI60710, Disperse Red 15), 1-hydroxy-4-[(4-methyl-2-sulphophenyl)-amino]-9,10-anthraquinone, 7-beta-D-glucopyranosyl-9,10-dihydro-1-methyl-9,10-dioxo-3,5,6,8-tetrahydroxy-2-anthracenecarboxylic acid (CI75470, Natural Red 4), 1-[(3-aminopropyl)amino]-9,10-anthraquinone (HC Red No. 8), 1,4-diamino-9,10-anthraquinone (CI61100, Disperse Violet No. 1), 1-amino-4-(methylamino)-9,10-anthraquinone (CI61105, Disperse Violet No. 4, Solvent Violet No. 12), N-(6-((3-chloro-4-(methylamino)phenyl)imino)-4-methyl-3-oxo-1,4-cyclohexadien-1-yl)urea (HC Red No. 9), 2-((4-(di(2-hydroxyethyl)-amino)phenyl)amino)-5-((2-hydroxyethyl)-amino)-2,5-cyclohexadiene-1,4-dione (HC Green No. 1), 2-hydroxy-1,4-naphthoquinone (CI75480, Natural Orange No. 6), 1,2-dihydro-2-(1,3-dihydro-3-oxo-2H-indol-2-ylidene)-3H-indol-3-one (CI73000), 1,3-bis(dicyanomethylene)indane, di[4-(diethylamino)phenyl]-[4-(ethylamino)-naphthyl]carbenium chloride (CI42595; Basic Blue No. 7), di[4-(dimethylamino)phenyl][4-(phenylamino)naphthyl]carbenium chloride (CI44045; Basic Blue No. 26), Basic Blue No. 77, 8-amino-2-bromo-5-hydroxy-4-imino-6-[(3-(trimethylammonio)phenyl)amino]-1(4H)-naphthalinone chloride (CI56059; Basic Blue No. 99), tri(4-amino-3-methylphenyl)carbenium chloride (CI42520; Basic Violet No. 2), di(4-aminophenyl)(4-amino-3-methylphenyl)carbenium chloride (CI42510; Basic Violet No. 14), 1-[(4-aminophenyl)azo]-7-(trimethylammonio)-2-naphthol chloride (CI 12250; Basic Brown No. 16), 3-[(4-amino-2,5-dimethoxyphenyl)azo]-N,N,N-trimethyl-benzenaminium chloride (CI112605, Basic Orange No. 69), 1-[(4-amino-2-nitrophenyl)azo]-7-(trimethylammonio)-2-naphthol chloride (Basic Brown No. 17), 1-[(4-amino-3-nitrophenyl)azo]-7-(trimethylammonio)-2-naphthol chloride (CI12251; Basic Brown No. 17), 2-((4-aminophenyl)azo)-1,3-dimethyl-1H-imidazol-3-ium chloride (Basic Orange No. 31), 3,7-diamino-2,8-dimethyl-5-phenylphenazinium chloride (CI50240; Basic Red No. 2), 1,4-dimethyl-5-[(4-(dimethylamino)phenyl)azo]-1,2,4-triazolium chloride (CI1055; Basic Red No. 22), 1,3-dimethyl-2-((4-dimethylamino)phenyl)azo-1H-imidazol-3-ium chloride (Basic Red No. 51), 2-hydroxy-1-[(2-methoxyphenyl)azo]-7-(trimethylammonio)naphthalene chloride (CI12245; Basic Red No. 76), 3-methyl-1-phenyl-4-[(3-(trimethylammonio)phenyl)azo]-pyrazol-5-one chloride (CI12719; Basic Yellow No. 57), 1-methyl-4-((methylphenyl-hydrazono)methyl)pyridinium methylsulphate (Basic Yellow No. 87), 1-(2-morpholiniumpropylamino)-4-hydroxy-9,10-anthraquinone methylsulphate, 1-[(3-(dimethylpropylaminium)propyl)amino]-4-(methylamino)-9,10-anthraquinone chloride, 1-[di(2-hydroxyethyl)amino]-3-methyl-4-[(4-nitrophenyl)azo]benzene (CI11210, Disperse Red No. 17), 1-[di(2-hydroxyethyl)amino]-4-[(4-nitrophenyl)azo]benzene, (Disperse Black No. 9), 4-[(4-aminophenyl)azo]-1-[di(2-hydroxyethyl)amino]-3-methylbenzene, (HC Yellow No. 7), 2,6-diamino-3-[(pyridin-3-yl)azo]pyridine and 2-((4-(ethyl(2-hydroxyethyl)amino)-2-methylphenyl)azo)-5-nitro-1,3-thiazole (CI111935; Disperse Blue No. 106), 3-(2′,6′-diamino-pyridyl-3′-azo)pyridine (=2,6-diamino-3-((pyridin-3-yl)azo)pyridine, N,N-di(2-hydroxyethyl)-3-methyl-4-((4-nitrophenyl)azo)aniline (Disperse Red 17, CI 11210), 3-diethylamino-7-(4-dimethylaminophenylazo)-5-phenylphenazinium chloride (CI 11050), 4-(2-thiazolylazo)resorcinol, 4-((4-phenylamino)azo)benzosulphonic acid sodium salt (Orange IV), 1-((3-aminopropyl)amino)-9,10-anthracenedione, (HC Red No. 8), 3′,3″,4,5,5′,5″,6,7-octabromophenol sulphonephthalein (Tetrabromophenol Blue), 1-((4-amino-3,5-dimethylphenyl)-(2,6-dichlorophenyl)methylene)-3,5-dimethyl-4-imino-2,5-cyclohexadiene phosphoric acid (1:1) (Basic Blue 77), 3′,3″,5′,5″-tetrabromo-m-cresol sulphonephthalein, 2,4-dinitro-1-naphthol-7-sulphonic acid disodium salt (Acid Yellow 1, CI 10316), 4-[2′-hydroxy-1′-naphthyl)azo]benzosulphonic acid sodium salt (Acid Orange 7, CI 15510), 3′,6′-dihydroxy-2′,4′,5′,7′-tetraiodospiro-[isobenzofuran-1(3H), 9′(9H)-xanthen]-3-one disodium salt (Acid Red 51, CI 45430), 6-hydroxy-5-((2-methoxy-5-methyl-4-sulphophenyl)azo)-2-naphthalenesulphonic acid disodium salt (FD&C Red 40, CI 16035), 2,4-dinitro-1-naphthol sodium salt (Acid Yellow 24; ClI 10315), 2′,4′,5′,7′-tetrabromo-4,5,6,7-tetrachloro-3′,6′-dihydroxyspiro-(isobenzofuran-[(3H), 9′[9H]xanthen]-3-one disodium salt (Acid Red 92; CI 45410), 4-(2-hydroxy-1-naphthylazo)-3-methyl-benzenesulphonic acid sodium salt (Acid Orange 8, CI 15575), 2-amino-1,4-naphthalenedione, dithizone (1,5-diphenylthiocarbazone), N-(2-hydroxyethyl))-2-nitro-4-trifluoromethyl)aniline (HC Yellow 13), N-(2-hydroxyethyl)-4-nitroaniline and 4-chloro-N-(2,3-dihydroxypropyl)-2-nitroaniline.

The abovementioned direct dyes may be present in a total amount of from about 0.01 to 4 percent by weight, where the total content of dyes in the colorant according to the invention is preferably about 0.01 to 10 percent by weight, in particular 0.1 to 5 percent weight.

In addition, the colorants according to the invention can also comprise naturally occurring dyes, such as, for example, henna red, henna neutral, henna black, camomile, sandalwood, black tea, buckthorn bark, sage, logwood, madder root, catechu, sedre and alkanna root.

The colorants according to the invention produce intense colorations even at physiologically compatible temperatures of less than 45° C. They are therefore particularly suitable for colouring human hair. For use on human hair, the colorants are usually incorporated into a hydrous cosmetic carrier. Suitable cosmetic carriers are, for example, creams, emulsions, gels or else surfactant-containing foaming solutions, such as, for example, shampoos or other preparations which are suitable for application to keratin-containing fibres. If necessary, it is also possible to incorporate the colorants into anhydrous carriers, powders, pellets or granules.

The colorant according to the invention can further comprise all additives which are customary and known for such preparations, for example perfume oils, complexing agents, waxes, preservatives, thickeners, antioxidants, alginates, guar gum, haircare substances, such as, for example, cationic polymers or lanolin derivatives, or anionic, nonionic, zwitterionic, amphoteric or cationic surface-active substances (surfactants). Preferably, amphoteric or nonionic surface-active substances are used, for example betaine surfactants, propionates and glycinates, such as, for example, cocoamphoglycinates or cocoamphodiglycinates, ethoxylated surfactants with 1 to 1000 ethylene oxide units, preferably with 1 to 300 ethylene oxide units, such as, for example, glyceride alkoxylates, for example castor oil ethoxylated with 25 ethylene oxide units, polyglycolamides, ethoxylated alcohols and ethoxylated fatty alcohols (fatty alcohol alkoxylates) and ethoxylated fatty acid sugar esters, in particular ethoxylated sorbitan fatty acid esters. The abovementioned constituents are used in the amounts customary for such purposes, for example the surface-active substances in a concentration of from 0.1 to 30 percent by weight, and the care substances in an amount of from 0.1 to 5 percent by weight.

The colorant according to the invention can, particularly if it is a hair colorant, be in the form of a powder or of granules, which is/are dissolved prior to application in an aqueous or aqueous-alcoholic preparation, or in the form of an aqueous or aqueous-alcoholic solution, a cream, a gel, an emulsion or an aerosol foam, where the hair colorant can be formulated either in the form of a single-component preparation or else in the form of a multicomponent preparation, for example in the form of a two-component preparation, in which case the particular oxime derivatives of the general formula A1 to A10 and the corresponding carbonyl compounds of the general formulae B1 to B12 are packaged separately from the other constituents (e.g., the ascorbic acid) and the ready-to-use hair colorant is only prepared immediately prior to application by mixing the two components.

The colorant according to the invention generally has a pH of from about 2 to 6, preferably about 3 to 5. Both organic and also inorganic acids are suitable for adjusting the pH according to the invention. Examples of suitable acids are the following acids: α-hydroxycarboxylic acids, such as, for example, glycolic acid, lactic acid, tartaric acid, citric acid or malic acid, gluconolactone, acetic acid, hydrochloric acid or phosphoric acid, and mixtures of these acids. Particular preference here is given to the use of the ascorbic acid used as reducing agent.

The colorant according to the invention is generally used by applying to the hair an amount of the hair colorant adequate for the hair colouring, about 30 to 120 grams depending on the length of hair, leaving the hair colorant to act at about 15 to 45 degrees Celsius for about 1 to 60 minutes, preferably 5 to 30 minutes, then thoroughly rinsing the hair with water, optionally washing with a shampoo and/or after-treating with a hair-conditioning composition and finally drying.

The colorant described above can also comprise natural or synthetic polymers or modified polymers of natural origin customary for cosmetic compositions, through which setting of the hair is achieved at the same time as the colouring. Such compositions are generally referred to as tinting setting compositions or colour setting compositions.

Of the synthetic polymers known for this purpose in cosmetics, mention may be made, for example, of polyvinylpyrrolidone, polyvinyl acetate, polyvinyl alcohol or polyacrylic compounds, such as polyacrylic acid or polymethacrylic acid, basic polymers of esters of polyacrylic acid, polymethacrylic acid and aminoalcohols, for example their salts or quaternization products, polyacrylonitrile, polyvinyl acetates and copolymers of such compounds, such as, for example, polyvinylpyrrolidone-vinyl acetate; whereas natural polymers or modified natural polymers which may be used are, for example, chitosan (deacetylated chitin) or chitosan derivatives. The abovementioned polymers may be present in the colorant according to the invention in the amounts customary for such compositions, in particular in an amount of from about 1 to 5 percent by weight.

The hair colorant with additional setting is used in a known and customary manner by wetting the hair with the setting composition, fixing (arranging) the hair in the hairstyle and then drying.

The colorant according to the invention permits an even, intense and long-lasting coloration of keratin fibres (for example human hair, wool or furs) without noteworthy discoloration of the skin and/or scalp.

The examples below are intended to illustrate the subject-matter of the invention in more detail without limiting it thereto.

### EXAMPLES

### Example 1

### Synthesis of 4-(dimethylamino)-1-(2-{[(1,3-dioxo-1,3-dihydro-2H-inden-2-ylidene)amino]oxy}-2-oxoethyl)pyridinium bromide

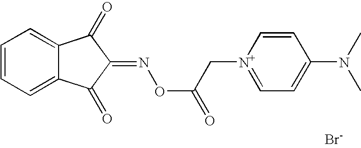

0.5 g (2.85 mmol) 1H-indene-1,2,3-trione 2-oxime were dissolved in 10 ml acetonitrile. The mixture was cooled to 0° C. followed by addition of 1.02 g (3.71 mmol) bromoacetic anhydride. The resulting suspension was then stirred for 2 hours under reflux until a yellow solution was obtained. TLC analysis showed complete consumption of the starting material. Then the reaction mixture was cooled to room temperature. 3.65 g (28.55 mmol)

4-dimethylamino pyridine were added in smalll portions while the mixture turned brown. After further cooling in an ice bath a brown residue was formed which was collected by filtration. The crude product was treated for 30 minutes with acetone at room temperature, filtered and dried in vacuum.

Yield: 1.15 g (96.3%)

¹H NMR (d₆-DMSO/300 MHz): δ=3.20 (s, 6H, 2 CH₃), 4.70 (s, 2H, CH₂), 6.86 (d, 2H, J=6 Hz, pyridine), 6.99 (d, 2H, J=7.2 Hz, pyridine), 8.15-8.21 (m, 4H, aromatic).

### Examples 2 to 6

### Hair Colorants

| | | |
|---|---|---|
| 5 | mmol | 4-(dimethylamino)-1-(2-{[(1,3-dioxo-1,3- |
| | | dihydro-2H-inden-2-ylidene)amino]oxy}-2- |
| | | oxoethyl)pyridinium bromide (=cationicoxime-ester |
| | | compound of the general formula A1 to A10) |
| 5 | mmol | carbonyl compounds of the general formula |
| | | B1 to B12 |
| 5.0 | g | ethanol |
| 4.0 | g | decylpolyglucose |
| 0.2 | g | ethylenediaminotetraacetic acid disodium |
| | | salt hydrate |
| ad 100.0 | g | water, demineralized |

The hair colouring is carried out by applying an amount of the colorant and of the reducing agent ascorbic acid (preferably 1-4 g/10 ml of coloring solution) adequate for the hair colouring to the hair.

After a contact time of 30 minutes at 40° C., the hair is rinsed with lukewarm water and dried.

The coloring results are summarized in Table 1 below.

TABLE 1

| Ex. No. | Carbonyl compound | Coloring result |
|---|---|---|
| 2 | 4-hydroxy-3-methoxybenzaldehyde | flax/fawn |
| 3 | 4-(dimethylamino)benzaldehyde | straw yellow |
| 4 | 4-methoxy-1-naphthaldehyde | old rose |
| 5 | (2E)-3-[4-dimethylamino)phenyl]-2-propenal | rust red |
| 6 | 2,4,6-trihydroxybenzaldehyde | orange |

Unless otherwise indicated, all percentages are by weight.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An agent for colouring keratin-containing fibres, comprising a) ascorbic acid, b) at least one cationic oxime-ester compound of the formula A1 to A10; 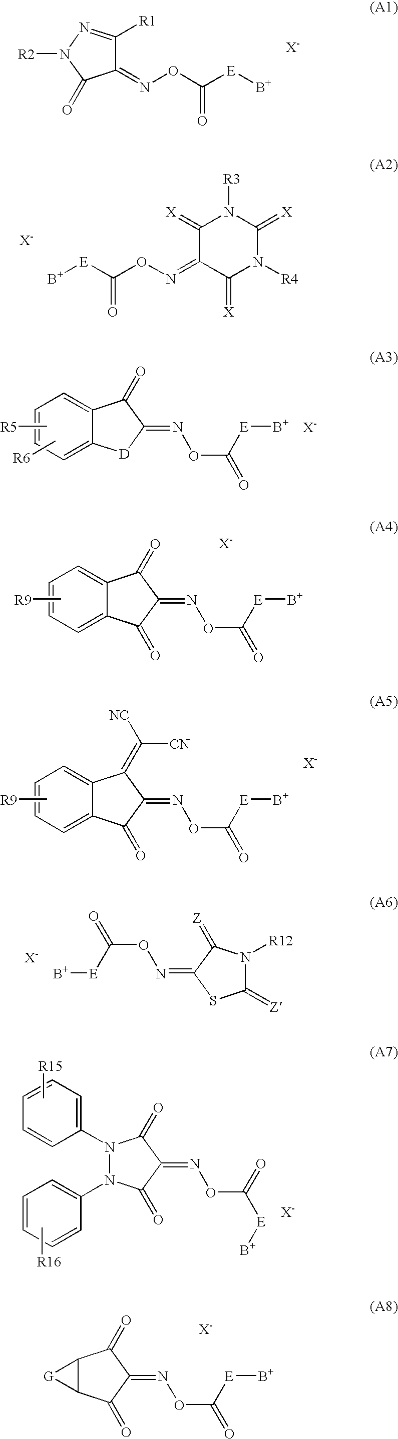 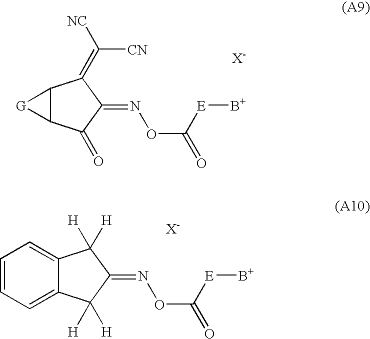
in which
R1 and R2 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group, a substituted or unsubstituted aminoaryl group, where R1 may be a C₁-C₆-alkoxy group.
E may be a C₁-C₄-alkyl bridge, a C₃-C₆-cycloalkyl bridge or an aryl bridge. B+may be
A) an aromatic heterocyclic ammonium compound; or
B) a non-aromatic heterocyclic ammonium compound; or
C) an alkylammonium compound or an arylammonium compound according to the formula —N⁺RₐR_{b}R_{c}, in which Rₐ, R_{b} and R_{c}, independently of one other, are a benzyl rest, a phenyl rest or a C₁-C₆-alkyl rest, whereas the prementioned alkyl groups may be unsubstituted or substituted with one or more hydroxy groups or amino groups; or
D) a cationic phosphonium group; and
X⁻ is a counterion;
R3 and R4 may be identical or different and, independently of one another, are a hydrogen atom or a substituted or unsubstituted C₁-C₆-alkyl group;
Y, Y′ and Y″, independently of one another, are an oxygen atom, a sulphur atom or an NH group;
D is an oxygen atom, a sulphur atom, a sulphoxyl group, a sulphonyl group or a group N—R₅ₐ, where R₅ₐ is a hydrogen atom or a substituted or unsubstituted C₁-C₆-alkyl group;
R5 and R6, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR7R8, where R7 and R8, independently of one another, are a hydrogen atom or a C₁-C₆-alkyl group;
R9 is a hydrogen atom, a halogen atom, a hydroxyl group, a nitro group, a C₁-C₆-alkyl group, a C₁-C₆-alkoxy group, a carboxyl group, a carboxamide group, a cyano group or an amino group —NR10R11, where R10 and R11, independently of one another, are a hydrogen atom or a C₁-C₆-alkyl group;
Z is an oxygen atom or a group —NR13, where R13 is a hydrogen atom or a C₁-C₆-alkyl group;
Z′ is a sulphur atom or a group —NR14, where R14 is a hydrogen atom or a C₁-C₆-alkyl group; and
R12 is a hydrogen atom, a C₁-C₆-alkyl group or a C₁-C₄-carboxyalkyl group;
R15 and R16 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR17R18, where R17 and R18, independently of one another, are a hydrogen atom or a C₁-C₆-alkyl group;
G is a substituted or unsubstituted, fused aromatic or heteroaromatic ring to which a further aromatic or heteroaromatic ring may additionally be fused;
and c) at least one reactive carbonyl compound of the formula B1 to B12; 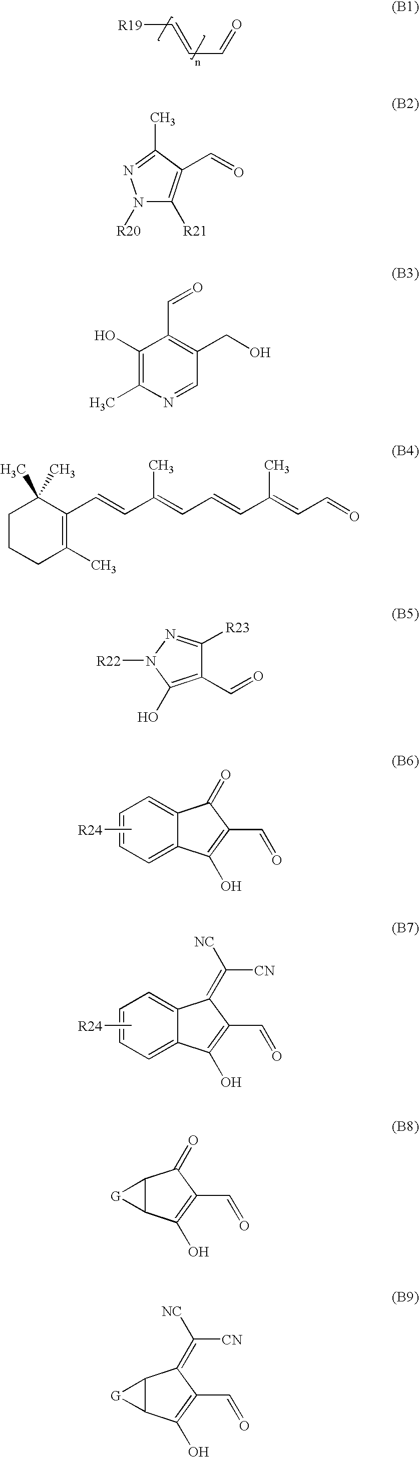 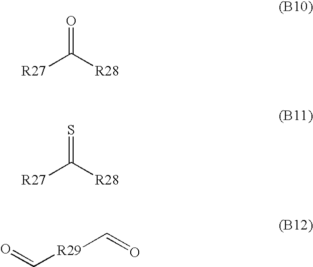
in which
R19 is a substituted or unsubstituted aryl group to which a further aromatic ring may optionally also be fused, or a substituted or unsubstituted, saturated or unsaturated heterocycle; and n is 0, 1, 2 or 3;
R20 is a C₁-C₆-alkyl group, a substituted or unsubstituted aryl radical or a substituted or unsubstituted, saturated or unsaturated heterocycle; and
R21 is a pyrrole radical, an imidazole radical, a pyrazole radical, an indole radical, a pyrrolidine radical, a morpholine radical, a dimethylamine radical, a phenol radical or a thiophenol radical, where these radicals are in each case bonded to the general formula B2 via the heteroatom;
R22 and R23 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group or a substituted or unsubstituted aminoaryl group, and R22 may be a C₁-C₆-alkoxy group;
R24 is a hydrogen atom, a halogen atom, a hydroxyl group, a nitro group, a C₁-C₆-alkyl group, a C₁-C₆-alkoxy group, a carboxyl group, a carboxamide group, a cyano group or an amino group —NR25R26, where R25 and R26, independently of one another, are a hydrogen atom or a C₁-C₆-alkyl group;
R27 and R28 may be identical or different and, independently of one another, are a C₁-C₆-alkyl group or a substituted or unsubstituted aryl group;
R29 is a substituted or unsubstituted C₁-C₆-alkyl bridge, a C₁-C₆-alkylene bridge or a substituted or unsubstituted aryl bridge
and G has the abovementioned meaning.

2. An agent according to claim 1, wherein the cationic oxime-ester compound of the general formula A1 to A10 is selected from the group consisiting of;
3-[4-({[(1,3-dioxo-1,3-dihydro-2H-inden-2-ylidene)amino]oxy}carbonyl)-benzyl]-1-methyl-1H-imidazol-3-ium chloride,
4-(dimethylamino)-1-(2-{[(1,3-dioxo-1,3-dihydro-2H-inden-2-ylidene)-amino]oxy}-2-oxoethyl)pyridinium bromide,
4-(dimethylamino)-1-[2-({[(2E)-4-nitro-1,3-dioxo-1,3-dihydro-2H-inden-2-ylidene]amino}oxy)-2-oxoethyl]pyridinium bromide,
3-(2-{[(1,3-dioxo-1,3-dihydro-2H-inden-2-ylidene)amino]oxy}-2-oxoethyl)-1-methyl-1H-imidazol-3-ium bromide,
1-methyl-3-[2-({[(2E)-4-nitro-1,3-dioxo-1,3-dihydro-2H-inden-2-ylidene]-amino}oxy)-2-oxoethyl]-1H-imidazol-3-ium bromide,
4-(dimethylamino)-1-[2-({[(2E)-5-methoxy-1,3-dioxo-1,3-dihydro-2H-inden-2-ylidene]amino}-oxy)-2-oxoethyl]pyridinium bromide,
4-(dimethylamino)-1-[2-({[(4E)-3-methyl-5-oxo-1-phenyl-1,5-dihydro-4H-pyrazol-4-ylidene]amino}oxy)-2-oxoethyl]pyridinium bromide,
4-(dimethylamino)-1-(2-oxo-2-{[(4,5,6,7-tetrachloro-1,3-dioxo-1,3-dihydro-2H-inden-2-ylidene)amino]oxy}ethyl)pyridinium bromide and
1-(2-{[((5Z)-1,3-diethyl-4-hydroxy-6-oxo-2-thioxotetrahydro-5(2H)-pyrimidinylidene)amino]oxy}-2-oxoethyl)-4-(dimethylamino)pyridinium bromide.

3. An agent according to claim 1, wherein the reactive carbonyl compound of the general formulae B1 to B12 is selected from the group consisting of benzaldehyde, 4-methoxybenzaldehyde, 4-hydroxybenzaldehyde, 3,4-dimethoxybenzaldehyde, 2,5-dimethoxybenzaldehyde, 3-hydroxy-4-methoxybenzaldehyde, 4-nitrobenzaldehyde, 4-hydroxy-3-methoxy-benzaldehyde, 4-(dimethylamino)benzaldehyde, (2E)-3-[4-(dimethylamino)phenyl]-2-propenal, 2,4,6-trihydroxybenzaldehyde, 3-hydroxy-5-(hydroxymethyl)-2-methylisonicotine aldehyde, all-trans-retinal, (2E,4E)-5-phenyl-2,4-pentadienal, pentanedial, 4-(methylsulphanyl)-benzaldehyde, 1-naphthaldehyde, (2E)-3-(4-nitrophenyl)-2-propenal, 3-phenoxybenzaldehyde, terephthalaldehyde, 3-hydroxy-1-oxo-1H-cyclopenta[a]-naphthalene-2-carbaldehyde, 3-hydroxy-1-oxo-1H-cyclopenta[b]-quinoxaline-2-carbaldehyde, 3-hydroxy-4-methyl-1-oxo-1,3a,4,8b-tetrahydrocyclopenta[b]indole-2-carbaldehyde, 4-hydroxy-6-oxo-6H-cyclopenta[b]thiophene-5-carbaldehyde, 3-hydroxy-5-nitro-1-oxo-1H-indene-2-carbaldehyde, 4,6-dichloro-3-hydroxy-1-oxo-1H-indene-2-carbaldehyde, 7-hydroxy-5-oxo-5H-cyclopenta[c]pyridine-6-carbaldehyde, 7-hydroxy-5-oxo-5H-cyclopenta[b]pyridine-6-carbaldehyde, 3-hydroxy-1-oxo-1H-indene-2-carbaldehyde, 5-(dimethylamino)-3-hydroxy-1-oxo-1H-indene-2-carbaldehyde, 3-hydroxy-4,5-dimethoxy-1-oxo-1H-indene-2-carbaldehyde, 3-hydroxy-5-methoxy-1-oxo-1H-indene-2-carbaldehyde, 3-methyl-5-(4-morpholinyl)-1-phenyl-1H-pyrazole-4-carbaldehyde, 3-methyl-1-phenyl-5-(1H-pyrrol-1-yl)-1H-pyrazole-4-carbaldehyde, 3-methyl-1-phenyl-5-(1-pyrrolidinyl)-1H-pyrazol-4-carbaldehyde, 3-methyl-1-(2-pyridinyl)-5-(1-pyrrolidinyl)-1H-pyrazole-4-carbaldehyde, 3-methyl-1-(2-pyridinyl)-5-(1H-pyrrol-1-yl)-1H-pyrazole-4-carbaldehyde, 4-methoxy-1-naphthaldehyde, 1H-indole-3-carbaldehyde, bis[4-(di-methylamino)-phenyl]methanethione, 2,4-dihydroxybenzaldehyde, 1-methyl-1H-indole-3-carbaldehyde and 3,4-dihydroxybenzaldehyde.

4. An agent according to claim 1, comprising from 0.1 to 10 percent by weight the oxime compound of the general formula A1 to A10 and the reactive carbonyl compound of the general formula B1 to B12.

5. An agent according to claim 1, additionally comprising one or more customary direct dyes from the group of acidic and basic dyes, nitro dyes, azo dyes, anthraquinone dyes and triphenylmethane dyes.

6. An agent according to claim 1, wherein the agent is a hair colorant.

7. An agent according to claim 1, additionally comprising at least one polymer which is customary for cosmetic compositions and which is selected from the group consisting of natural polymers, synthetic polymers, modified polymers of natural origin, and mixtures thereof, and is in the form of a tinting setting composition or colour setting composition.

8. A method of colouring hair in which a colorant according to claim 1 is applied to the hair in an amount adequate for the hair colouring, about 30 to 120 grams, the hair colorant is left to act at about 15 to 45 degrees Celsius for about 1 to 60 minutes, and the hair is then thoroughly rinsed with water, and finally dried.

9. A method of colouring hair according to claim 8, wherein the hair is washed with a shampoo or after-treated with a hair-conditioning composition before dryeing.

10. A method of colouring hair according to claim 8, wherein the hair is washed with a shampoo and subsequently after-treated with a hair-conditioning composition before dryeing.
